SEQUENCE LISTING (8 sequences)

SEQ ID NO: 1 - DNA, 2035 bp, synthetic construct
  source 1..2035: mol_type = other DNA
aatgggctcggtcccagtaacttgtttggtttaacgcagtacaattattgcacagccttcttcgatggaggagcacctttgtctcaaggtgctggatatggcatggtgtgcttgttcggagttgtcttttcattcgttacatcagctttgatttggcttgagtggagatttgcaggggccaatcactcatcggaagcattcaacactgctgggcgcactgtcaaaacagggctgattgcgaacgttatcgtttctcagtggacttgggcagcaactttactccaatcttcgaatgtggcttggcagtatggcatttcaggaccgttctggtatgcttcaggagctaccattcaaatcattcttttcggaatccttgcggttgaaatcaaacgaaaggctccaaacgctcacactctattagaaattcttcgtgctcgctggggtaatacagctcataaggttttctttgtttttgcaatcatgacaaatattattgtgacatctatgttgttgcttggaggagcatccgtcatgaatgccttgacaggaatgaatttgtatgctgcttgcattctgatacctgttggagtcattctgtacactgctgttggaggtttaaaggctacatttatggcatcctgtgtgaattcattttatgttctcgtcatgctctgcttattttcattagctgtttatgccggaactgactccatcttgggctcacctgcaaaggtgtacgacaacttgcaaacatttgtcggtactccagcaccttctaatgctactgaatattctgaaattgctgatttgaagttggggccagttgctgataacaaagatggatcttttgtgactatgttttcagaaggtggattcatctttggtattatcaatatcattggtaactttggtaccgtttttgtggatcaatcttactacatgggtgcaatagcttcgagaccttctgcgtcttggaaaggttatatccttggtggcttgatgtggttctcgattccatttactcttgctacatctcttggtcttgcctctagagctgccgggcttccctgaatgctagtgaagctggttcagggcttgtaccgcctgcaactgcagtgtacctctttggaaatggaggtgcatggcttattgctctcatgcttctaatggctgtcacatctactgccaattcagagctaatcgctgtctcatcccttttctcctacgatatttataggacatacataaatcctaaagctactggaaaacagatagtgagaatttcccgtgcgatgattgttgtttttggtctcttcatgggttgcatggcaataatccttttgaaatcggtttaagcttaggttgggtatacctctttatgggcattgccattggtggagctgttttcccaatctatgcctctttaacttggaagaaggcaagtgctgcaggtgcaatgatcggtgctattgttggaactatcagtggctttatcacatggctggtagtatgccaaggatattacggatcaattaatgtagataatcttggtggtaactatcctatgctcggtggaaaccttgcatccatttttgtttccatgatagtgtgctctgcaatttctttgtataaaccacaggactatgactggcaaacgactcgagaaattgctcttgttgaagaggatggtgttccaaaagaagaagaacagagtgctcaagactccaggaagctatggatagggcagcaaagtatatgaaatgggcatgttggggattgactctcatcttgattgtagcttggcctttattgacactccctgcgggtgtcttcagcaagggttatttcacgttttgggtaatactttcaataatttggggattgcttgctactgcaattggattttctttaccattttgggaatcccgggatgctatttacagcatcgtcatgggtctcatcagttgctctccaaacccagaagttccagttactggtgcagcacaatccttcccttcaccttcatttggaaagc

SEQ ID NO: 2 - DNA, 2034 bp, synthetic construct
  source 1..2034: mol_type = other DNA
aatgggctcggtcccagtaacttgtttggtttaacgcagtacaattattgcacagccttcttcgatggaggagcacctttgtctcaaggtgctggatatggcatggtgtgcttgttcggagttgtcttttcattcgttacatcagctttgatttggcttgagtggagatttgcaggggccaatcactcatcggaagcattcaacactgctgggcgcactgtcaaaacagggctgattgcgaacgttatcgtttctcagtggacttgggcagcaactttactccaatcttcgaatgtggcttggcagtatggcatttcaggaccgttctggtatgcttcaggagctaccattcaaatcattcttttcggaatccttgcggttgaaatcaaacgaaaggctccaaacgctcacactctattagaaattcttcgtgctcgctggggtaatacagctcataaggttttctttgtttttgcaatcatgacaaatattattgtgacatctatgttgttgcttggaggagcatccgtcatgaatgccttgacaggaatgaatttgtatgctgcttgcattctgatacctgttggagtcattctgtacactgctgttggaggtttaaaggctacatttatggcatcctgtgtgaattcattttatgttctcgtcatgctctgcttattttcattagctgtttatgccggaactgactccatcttgggctcacctgcaaggtgtacgacaacttgcaaacatttgtcggtactccagcaccttctaatgctactgaatattctgaaattgctgatttgaagttggggccagttgctgataacaaaggtggatcttttgtgactatgttttcagaaggtggattcatctttggtattatcaatatcattggtaactttggtaccgtttttgtggatcaatcttactacatgggtgcaatagcttcgagaccttctgcgtcttggaaaggttatatccttggtggcttgatgtggttctcgattccatttactcttgctacatctcttggtcttgcctctagagctgccgggcttcccctgaatgctagtgaagctggttcagggcttgtaccgcctgcaactgcagtgtacctctttggaaatggaggtgcatggcttattgctctcatgcttctaatggctgtcacatctactgccaattcagagctaatcgctgtctcatcccttttctcctacgatatttataggacatacataaatcctaaagctactggaaaacagatagtgagaatttcccgtgcgatgattgttgtttttggtctcttcatgggttgcatggcaataatccttttgaaatcggtttaagcttaggttgggtatacctctttatgggcattgccattggtggagctgttttcccaatctatgcctctttaacttggaagaaggcaagtgctgcaggtgcaatgatcggtgctattgttggaactatcagtggctttatcacatggctggtagtatgccaaggatattacggatcaattaatgtagataatcttggtggtaactatcctatgctcggtggaaaccttgcatccatttttgtttccatgatagtgtgctctgcaatttctttgtataaaccacaggactatgactggcaaacgactcgagaaattgctcttgttgaagaggatggtgttccaaagaagaagaacagagtgctcaagactccaggaagctatggatagggcagcaaagtatatgaaatgggcatgttggggattgactctcatcttgattgtagcttggcctttattgacactccctgcgggtgtcttcagcaagggttatttcacgttttgggtaatactttcaataatttggggattgcttgctactgcaattggattttctttaccattttgggaatcccgggatgctatttacagcatcgtcatgggtctcatcagttgctctccaaacccagaagttccagttactggtgcagcacaatccttcccttcaccttcatttggaaagc

SEQ ID NO: 3 - DNA, 676 bp, synthetic construct
  source 1..676: mol_type = other DNA
ggcagatatggggacgttgttggagttttattgggtggcgagagggcagtgatcgtgtcagatccacatgtggcacaatatgtccttgtagatagaccagacgtgtttgtaaaatcaggaacagcattctttccggggtcaagtcttgctggtaatggtctattagtttccgacggtgaagtatggcgtcgccaagaagacttgcagccccgtctttccgaaaagctgctgtagagcgatacgcaacatcaatgattgactgcactcgtaaaaacctgttggatagaagaaagtggagacctggagcagtacgaaatgtttattcagacttcaatgaattgactctgcatataacattgaatgcactatttggttcggaggatatgtcttctgtaacagctcttgaggtaacatcttcaattcaacagtgtttcgaatattttgcccaacgctctgtaactgctttcattataccggagtccattccaacaccgagcaatttgaggttctcaagggcagtctcacgtttggataaagcagtgtatggcctgattgcttgccgaagaaagaatcttgagacgcagaattctcaattgtctgaagacaaattgcaaaaaggtgatttattgggatcgctaataactgcaacagatgaagatagcaccggcatggatga

SEQ ID NO: 4 - DNA, 677 bp, synthetic construct
  source 1..677: mol_type = other DNA
ggcagatatggggacgttgttggagttttattgggtggcgagagggcagtgatcgtgtcagatccacatctggcacaatatgtccttgtagatagaccagatgtgtttgtaaaatcaggaacagcattctttccggggtcaaatcttgctggtaatggtctattagtttccgacggtgaagtatggcgtcgccaaagaagacttgcagccccgtctttccgaaaagctgctgtagagcgatacgcaacatcaatgattgactgcactcgtaaaaatctgttggatagaagaaagtggagacctggcgcagtacgaaatgtttattcagacttcaatgaattgactctgcaaataacattgaatgcactatttggttcggaggatatgtcttctgtaacagctcttgaggtaacatcttcaattcaacagtgtttcgaatattttgcccaacgctctgtaactgctttcattataccggagtccattccaacaccgagcaatttgaggttctcaagggcagtctcacgtctggataaagcagtttatggcctgattgctcgccgaagaaagaatttagagacgcagaattctcgattgtctgaagatacattgcgaaaaggggatttattgggatcgctaatatctgcaacagatgaagatagcaccggcatggatga

SEQ ID NO: 5 - DNA, 1756 bp, synthetic construct
  source 1..1756: mol_type = other DNA
ccgttttctattactgccgccgtcatggagttgatggcgacctgtatcttcgtctatatcggtactggaacagcggtgacgttttcttcgtatgctactgatacgacgccattcactcgcgagtccattaccatcgaggacaatgatattcaatctggatttcaagttctgaacgatcgcttgacgatcaacggctcgtggggcgtgacgactgccttggcctttggattggctatcaccgtgctcgcatatgtacgtttgtttgtgcatcgctacgttcgatgttttggcgatgagaactgatgtgtgtgtttgagtattttggcccatggcatcgtggtgcgcccacgatcagggagatgcaaggagcatttggcatcgttaccgaggcacaagcttcgtgatctattttttccctttttctttttattttaagcaatgtcaagtcccaatatcattatcattatcattatcagcagcagcctaatcataatcatgtccatcggcatcatcaccaggctgtgtcacagatccatcatggacatcacacctaattgatgtaatttgtaatgctttgtgctaacgaaatgtttgtattgcgcaggcaacgtcccatctgtcgggcggccagctgaacccggtggtcagctttgctcttgtttgcgtgaaagctctttctcctttgcaaggcttggtgaacatttgctgtcaaattgtgagagccatccttgggtcgtccttcctgtacgcaacgatcccttcggaggacagaacaacgcttggttctaatgtcgtggctcctggggtgtcgaatatgaacgccttctgtggcgaggcggtgatgacgtttacgttggttttggttgtcctggaaacggccgtgaacaagaaatctgtcgctcgtgctcaggtgagaaagaacggaccgagagtccatctgttgtgtcttgaaagagatctgtgctttcctgttggatggactcttgtgggttgatccaagcttttgttcgttgcctgccaatgaaataacatgtcctgttgtcctcgcccacggtgaaaggatatgttccgtttgtttgaaggcgatttatccgttatggcagccatgaagccgaaagcaactggaggctcttcgggtgcatggtttacacgttttgttatggttgaactatatcgctgtttgtttttgttttttgcttttgcaggctcctttggcgatcggttttgccgtgttctgcggtcatgccgtattgcttccaattgacggttgctccatcgtaagcttgttgggtcgtggggtttcttggcgctgtcttgtgtttttggacctgttttggcttgtttatttttgttgttttcggtcttgtggtcgttggatctgcgtggctcgttgtgttggcgtctcgaaaatgcatgaacgcctgcctatttgttttccagcgaaggcgttgtatgatgctgtcgtctttgtaatttttgtgtgtgttttccgttgtgtgcagaaccctgctcgttcttttggtcctgcggttgtctctggcacctggaacgatttctgggtgtttgtgatcggtcccctcgtgggcacgatcgtcgctcttcctttccacttcttgttccgcagcaacatcgaagacattcctgcttctgatcctgttgacctccgtgcagcagcgagcatgaaagaagaaaaatgtgctgataagggacacacttccgaagctagtgacaaggtgcagcgtgctgtagaagacgg

SEQ ID NO: 6 - DNA, 1173 bp, synthetic construct
  source 1..1173: mol_type = other DNA
ccgttttctattactgccgccgtcgcggagttgatggcgacctgtatcttcgtctatatcggtactggaacagcggtgacgttttcttcgtatgctactgatacgacgccattcactcgcgagtccattaccatcgaggacaatgatattcaatctggatttcaagttctgaacgatcgcttgacgatcaacggctcgtgggcgtgacgactgccttggcctttggattggctatcaccgtgctcgcatatgcaacgtcccatctgtcgggcggccagctgaacccggtggtcagctttgctcttgtttgcgtgaaagctctttctccacaaggcttggtgaacatttgctgtcaaattgtgggagccatccttgggtcgtccttcctgtacgcaacgatcccttcggaggacagaacaacgcttggttctaatgtcgtggctcctggggtgtcgaatatgaacgccttctgtggcgaggcggtgatgacgtttacgttggttttggttgtcctggaaacggccgtgaacaagaaatctgtcgctcgtgctcaggtgagaaagaacggaccgagagtccatctgttgtgtcttgaaagagatctgtgctttcctgttggatggactcttgtgggttgatccaagcttttgttcgttgcctgccaatgaaataacatgtcctgttgtcctcgcccacggtgaaaggatatgttccgtttgtttgaaggcgatttatccgttatggcagccatgaagccgaaagcaactggaggctcttcgggtgcatggtttacacgttttgttatggttgaactatatcgctgtttgtttttgtttttgcttttgcaggctcctttggcgatcggttttgccgtgttctgcggtcatgccgtattgcttccaattgacggttgctccatcaaccctgctcgttcttttggtcctgcggttgtctctggcacctggaacgatttctgggttttgtgatcggtccctcgtgggcacgatcgtcgctcttcctttccacttcttgttccgcagcaacatcgaagacattcctgcttctgatcctgttgacctccgtgcagcagcgagcatgaaagaagaaaatgtgctgataagggacacacttccgaagctagtgacaaggtgcagcgtgctgtagaagacggta

SEQ ID NO: 7 - DNA, 1278 bp, synthetic construct
  source 1..1278: mol_type = other DNA
ttttctattactgccgccgtcgcggagttgatggcgacctgtatcttcgtctatatcggtactgggacagcggtgacgttttcttcgtatgctactgatacgacgccgttcactcgcgagtccattaccatcgaggacagtgatattcaatctggatttcaagttctgaacgatcgcttgacgatcaacggctcgtggggcgtgacgactgccttggcctttggattggctatcaccgtgctcgcatatgtacgtttgtttgtgcatcgctacgttcgatgttttggcgatgagaactgatgcgtgtgtttgagtattttggcctctcgcatcgtggcgcgccccacgatccagggagatgcaaggagcatttggcatcgttaccgacgcacacgtttcgtgatcttttatctttttcctttttttttaatgtcaagtcccaatatcattatcattatcatcatcagcataatcataaccatccatcggcatcaggctgtgtcacagatccatcatggacatcacacctgacaactaactgatgtaattcgtgctttgtgctaacgaaatgttttgtattgcgcaggcaacgtcccatctgtcgggcggccagctgaacccggtggtcagctttgctcttgtttgcgtgaaagctctttctccattgcaaggcttggtgaacatttgctgtcaaattgtgggagccatccttgggtcgtccttcctgtatgcaacgatcccttcggaggacagaacaacgcttggttctaatgtcgtggctcctggggtgtcgaatatgaacgccttctgtggcgaggcggtgatgacgtttacgttggttttggtcgtcctggaaacggccgtgaacaagaaatctgtcgctcgtgctcaggctcctttggcgatcggttttgccgtgttctgcggtcatgccgtattgcttccaattgacggatgctccatcaaccctgctcgttcttttggtcctgcggttgtctctggcacctggaacgatttctgggtgtttgtgatcggtcccctcgtgggcacgatcgtcgctcttcctttccacttcttgttccgcagcaacatcgaagacattcctgcttctgatcctgttgacctccgtgcagcagcgagcatgaaaaagaaaaatctactcatcacagggaatttcttgcggaagccagtcccagcatgcaacgtgttgcagaagaagctgaatccttggctgcaggaggtggattaatccctgaaggagcccaaacaaatgctgga

SEQ ID NO: 8 - DNA, 960 bp, synthetic construct
  source 1..960: mol_type = other DNA
cgttttctattactgccgccgtcgcggagttgatggcgacgtgtatcttcgtctatatcggtactggaacagcggtgacgttttcttcgtatgctactgatacgacaccattcactcgcgagtccattaccatcgaggacaatgatattcaatctggatttcaagttctgaacgatcgcttgacgatcaacggctcgtggggcgtgacgactgccttggccttcggattggctatcaccgtgctcgcatatgcaacgtcccatctgtcgggcggccagctgaacccggtggtcagctttgctcttgtttgcgtgaaagctctttctccattgcaaggcttggtgaacatttgctgtcaaattgtgggagccatccttgggtcgtccttcctgtacgcaacgatcccttcggaggacagaacaacgcttggttctaatgtcgtggctcctggggtgtcgaatatgaacgccttctgtggcgaggcggtgatgacgtttacgttggttttggtcgtcctggaaacggccgtgaacaagaaatctgtcgctcgtgctcaggctcctttggcgatcggttttgccgtgttctgcggtcatgccgtattgcttccaattgacggttgctccatcaaccctgctcgttcttttggtcctgcggttgtctctggcacctggaacgatttctgggtgtttgtgatcggtccctcgtgggcacgatcgtcgctcttcctttccacttcttgttccgcagcaacatcgaagacgttcctgcttctgatcctgttgacctccgtgcagcagcgagcatgaaaaagaaaaatctactcatcacagggaatttcttgccgaagccagtcccagcatgcaacgcgttgcagaagaagctgaatccttggctgcaggaggtggattaatccctgaaggagcccaaacaaatgctggaggcgtattactgccgccg

(19) United States
(12) Patent Application Publication
(10) Pub. No.: US 2023/0323279 A1
(43) Pub. Date: Oct. 12, 2023

(54) MUTANT ALGAE, METHOD OF PREPARATION AND APPLICATION THEREOF

(71) Applicant: Reliance Industries Limited, Mumbai (IN)

(72) Inventors: Chitranshu KUMAR, Thane (IN); Bhaskar BHADRA, Hyderabad (IN); Saranya KARUPPASAMY, Sivakasi (IN); Shradha KHATER, Mumbai (IN)

(21) Appl. No.: 18/183,087

(22) Filed: Mar. 13, 2023

(30) Foreign Application Priority Data
Mar. 16, 2022 (IN) 202221014245

(51) Int. Cl. C12N 1/12; C07K 14/405

(52) U.S. Cl. CPC C12N 1/12; C07K 14/405

(57) ABSTRACT

The present disclosure relates to mutant algae resistant to salicylanilide based drug. The mutant algae has improved productivity and improved photosynthetic efficiency as compared to wild type of the algae in presence of the salicylanilide based drug. The present disclosure further relates to a method of preparing said mutant algae.

### CROSS REFERENCE TO RELATED APPLICATION

This application claims priority to Indian Patent Application No. 202221014245, filed Mar. 16, 2022, the content of which is incorporated by reference in its entirety into the present disclosure.

### SEQUENCE LISTING

The instant application contains a Sequence Listing which has been submitted electronically in XML format (ST.26) and is hereby incorporated by reference in its entirety. Said XML copy, created on Jun. 13, 2023, is named 20XS-350615-US_ST26.xml and is 18.1 KB in size.

### TECHNICAL FIELD

The present disclosure relates to the field of cell biology and microbiology. Particularly, the present disclosure relates to mutant algae resistant to salicylanilide based drug and improved biotic stress tolerance. The mutant algae is noted to have improved productivity and improved photosynthetic efficiency as compared to wild type of the algae in presence of the salicylanilide based drug. The present disclosure further relates to a method of obtaining said mutant algae.

### BACKGROUND OF THE DISCLOSURE

Commercialization of diverse products from algae would require consistent production of desired material in required amounts at a large scale. This means algae crops must be cultivated at a large scale throughout the year with consistent high biomass productivity (yield/hector). Algal crop cultivated in the open ponds are highly susceptible to contamination by organisms that graze upon and feed on the crop or the culture is taken over by a foreign alga instead of the desired crop. This frequent predation and competition results in frequent culture crashes and crop loss thus threatening commercialization of products from algae.

Few strategies have been tried to alleviate the influence of grazers and contaminants on the algal crop. Selective growth conditions like maintaining high salinity, high alkalinity and high nutrition have been used to cultivate different algal strains at a commercial scale. Providing selective growth conditions is an effective strategy for managing grazers and contaminants, however, it limits the desired high productivity of the biomass. Hence, the selective growth condition strategy is not suitable for most of the algal cultivation requiring high biomass.

Further, chemical crop control strategy has been employed for preventing contaminants and predators of algae. Hypochlorite has been used to control protozoans in Nannochloropsis mass cultures. Ammonia and acidic pH shock have been used to control rotifers in open ponds. However, it is noted that chemical crop control strategy has not been successful at commercial scale as use of most chemicals has adverse effects on algae biomass productivity, toxic to environment, cost incompatible and in some cases are unable to effectively control the grazers and weeds.

Thus, there is a need for robust algal strains with improved biotic stress tolerance and productivity that can be cultivated at a commercial scale in open pond or in photobioreactor (PBR). Methods to develop such algae and identify the same are therefore required to benefit large scale cultivation of algae for commercialization of products. The present disclosure addresses said need.

### STATEMENT OF THE DISCLOSURE

Accordingly, the present disclosure describes mutant algae resistant to Salicylanilide based drugs. The mutant algae show improved biotic stress tolerance, provide for improved productivity and improved photosynthetic efficiency as compared to wild type of the algae in presence of salicylanilide based drug.

Particularly, the productivity of the mutant algae is improved by at least 30% and the photosynthetic efficiency of the mutant algae is improved by at least 2 fold as compared to wild type of the algae, respectively. The mutant algae further demonstrate improved nitrogen content by at least 10% as compared to wild type of the algae in presence of drug. The mutant algae has at least 2 fold reduced adenosine triphosphate (ATP) depletion as compared to 10 fold reduction in wild type of the algae upon being exposed to Salicylanilide class of drug.

The present disclosure further describes a method of obtaining the mutant algae described above, said method comprises: subjecting wild type algae to mutagenesis; culturing mutagen exposed live algae under dark and subsequently in low light conditions to obtain modified algae; and enriching the modified algae to obtain the algae resistant to salicylanilide based drug.

### BRIEF DESCRIPTION OF THE ACCOMPANYING FIGURES

The patent or application file contains at least one drawing executed in color. Copies of this patent or patent application publication with color drawing(s) will be provided by the Office upon request and payment of the necessary fee.

In order that the present disclosure may be readily understood and put into practical effect, reference will now be made to exemplary embodiments as illustrated with reference to the accompanying figures. The figures together with detailed description below, are incorporated in and form part of the specification, and serve to further illustrate the embodiments and explain various principles and advantages, where:

FIG. 1 A) illustrates a plot describing no growth of wild type of the algae (WT) in presence of Niclosamide (OD at 750 nm); and B) depicts an image of cultures showing no growth of wild type algae in presence of Niclosamide.

FIG. 2 A) illustrates a plot describing growth of mutant algae (NCAR-1) in different concentrations of Niclosamide (OD at 750 nm); and B) depicts an image of cultures showing growth of mutant algae (NCAR-1) at different concentrations of Niclosamide.

FIG. 3 A) illustrates a plot describing growth of mutant algae (NCAR-2) in different concentrations of Niclosamide (OD at 750 nm); and B) depicts an image of cultures showing growth of mutant algae (NCAR-2) at different concentrations of Niclosamide.

FIG. 4 A) illustrates a plot describing comparative growth of wild type of the algae and mutant algae (NCAR-1 and NCAR-2) at different concentrations of Niclosamide; and B) depicts an image of cultures showing no growth of wild type algae (WT) and growth of mutant algae (NCAR-1 and NCAR-2) at 10 ppm of Niclosamide, respectively.

FIG. 5 illustrates a plot describing volumetric productivity (AFDW mg/L/day) of wild type of the algae and mutant algae at different concentrations of Niclosamide.

FIG. 6 illustrate plots describing photosynthetic efficiency/health of- A) wild type of the algae (WT); B) mutant algae (NCAR-1); and C) mutant algae (NCAR-2) at different concentrations of Niclosamide (last day is 7^{th} Day).

FIG. 7 illustrate plots describing growth of wild type of the algae (WT) at different concentrations of- A) Niclosamide; B) Oxyclozanide; C) Rafoxanide; and D) Closantel.

FIG. 8 illustrate plots describing growth of mutant algae (NCAR-1) at different concentrations of- A) Niclosamide; B) Oxyclozanide; C) Rafoxanide; and D) Closantel.

FIG. 9 illustrate plots describing growth of mutant algae (NCAR-2) at different concentrations of- A) Niclosamide; B) Oxyclozanide; C) Rafoxanide; and D) Closantel.

FIG. 10 illustrates a plot describing volumetric productivity (mg/L/day) of wild type of the algae (WT) in presence of 2 ppm and 5 ppm of Niclosamide, Oxycycloxanide, Rafoxanide & Closantel, respectively.

FIG. 11 illustrates a plot describing volumetric productivity (mg/L/day) of mutant algae (NCAR-1) in presence of 2 ppm and 5 ppm of Niclosamide, Oxycycloxanide, Rafoxanide & Closantel, respectively.

FIG. 12 illustrates a plot describing volumetric productivity (mg/L/day) of mutant algae (NCAR-2) in presence of 2 ppm and 5 ppm of Niclosamide, Oxycycloxanide, Rafoxanide & Closantel, respectively.

FIG. 13 illustrates sequence alignment between a gene encoding (putative aquaporin) protein 1 of wild type of the algae (WT) and a gene encoding (putative aquaporin) protein 1 of mutant algae (NCAR1). Two long regions of insertion are highlighted in yellow; Other point insertions and base changes are marked with boxes.

FIG. 14 illustrates sequence alignment between a gene encoding (putative aquaporin) protein 2 of wild type of the algae (WT) and a gene encoding (putative aquaporin) protein 2 of mutant algae (NCAR1). Base changes and point insertions are shown in boxes; large insertion region is highlighted in yellow.

FIG. 15 illustrates sequence alignment between a gene encoding (putative proton symporter) protein 3 of wild type of the algae (WT) and a gene encoding (putative proton symporter) protein 3 of mutant algae (NCAR1). Base changes and insertion mutations are marked in boxes

FIG. 16 illustrates sequence alignment between a gene encoding (putative cytochrome P450) protein 4 of wild type of the algae (WT) and a gene encoding (putative cytochrome P450) protein 4 of mutant algae (NCAR1). The base changes and deletion are shown in boxes.

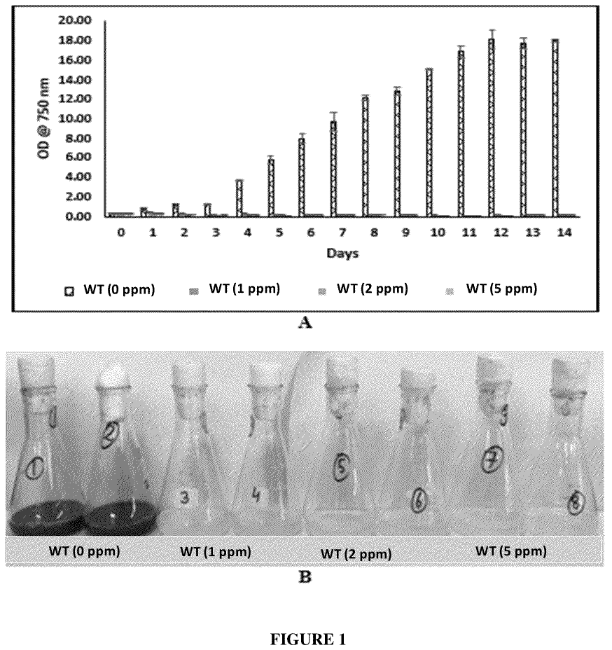

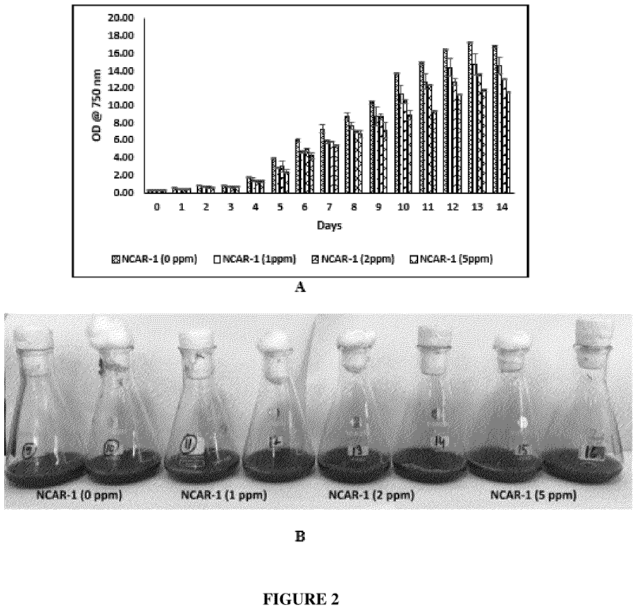

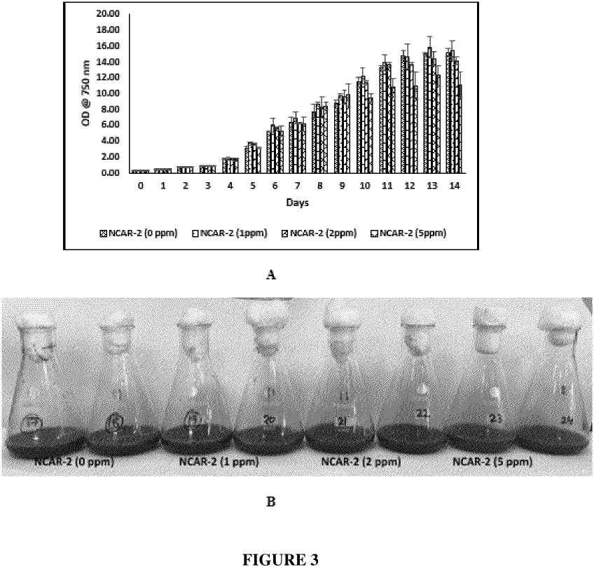

### DETAILED DESCRIPTION OF THE DISCLOSURE

Unless otherwise defined, all terms used in the disclosure, including technical and scientific terms, have meaning as commonly understood by one of ordinary skill in the art to which this disclosure belongs. By means of further guidance, term definitions are included for better understanding of the present disclosure.

As used herein, the singular forms ‘a’, ‘an’ and ‘the’ include both singular and plural referents unless the context clearly dictates otherwise.

The term ‘comprising’, ‘comprises’ or ‘comprised of’ as used herein are synonymous with ‘including’, ‘includes’, ‘containing’ or ‘contains’ and are inclusive or open-ended and do not exclude additional, non-recited members, elements or method steps.

The recitation of numerical ranges by endpoints includes all numbers and fractions subsumed within the respective ranges, as well as the recited endpoints.

The term ‘about’ as used herein when referring to a measurable value such as a parameter, an amount, a temporal duration, and the like, is meant to encompass variations of ± 10% or less, preferably ±5% or less, more preferably ±1% or less and still more preferably ±0.1% or less of and from the specified value, insofar such variations are appropriate to perform the present disclosure. It is to be understood that the value to which the modifier ‘about’ refers is itself also specifically, and preferably disclosed.

The term ‘and/or’ as used in a phares such as ‘A and/or B’ herein is intended to include ‘A and B’, ‘A or B’, ‘A’ and ‘B’.

Reference throughout this specification to ‘some embodiments’, ‘one embodiment’ or ‘an embodiment’ means that a particular feature, structure or characteristic described in connection with the embodiment may be included in at least one embodiment of the present disclosure. Thus, the appearances of the phrases ‘in some embodiments’, ‘in one embodiment’ or ‘in an embodiment’ in various places throughout this specification may not necessarily all refer to the same embodiment. It is appreciated that certain features of the disclosure, which are for clarity, described in the context of separate embodiments, may also be provided in combination in a single embodiment. Conversely, various features of the disclosure, which are, for brevity described in the context of a single embodiment, may also be provided separately or in any suitable sub-combination.

Generally, nomenclatures used in connection with, and techniques of biochemistry, enzymology, molecular and cellular biology, microbiology, genetics and protein/nucleic acid chemistry and hybridization described herein are those well-known and commonly used in the art. To facilitate an understanding of the present disclosure, a number of terms and phrases are defined below.

The terms ‘algae’, ‘algal cell’ or ‘algal strain’ as used herein refers to microalgae which are microscopic algae invisible to the naked eye. The microalgae are unicellular species which exist individually, in chains or in groups.

The term ‘mutant algae’ as used herein refers to mutant microalgae carrying mutations in the form of insertion and/or deletion of single nucleotide and/or multiple nucleotides leading to improved characteristics in the algae which is absent in the algae not having said mutations. The algae not having said mutation(s) is referred to herein as wild type algae or wild type of the algae. In other words, the term ‘wild type algae’ or ‘wild type of the algae’ as used herein refers to algae found in nature or naturally occurring without said mutation(s). The wild type algal cells employed in the present invention Is from the Applicant’s strain collection facility. The Picochlorum strain used was collected from Maharashtra coast at Karanja.

The term ‘biomass’ as used herein refers in general to organic matter produced by a biological cell. The biomass referred herein particularly relates to algae biomass. The algae biomass can be dry, substantially dry, or wet. ‘Biomass’ should be understood to include proteins, lipids, polysaccharide and/or other biomolecule, whether retained within the algae or excreted from the algae, in addition to other molecules synthesised by the algae.

The term ‘productivity’ as used herein refers to amount of product the algae can produce under defined conditions. Productivity can be measured in grams of dry weight of algae. Alternatively, productivity can be measured in terms of the rate of production of a desirable product or product(s) such as lipid, protein, carbohydrate and/or any other biomolecule produced by the algae. The term ‘volumetric productivity’ refers to increase in biomass weight of algae/L/day.

The term ‘photosynthetic efficiency’ as used herein refers to the fraction of light energy converted into chemical energy during photosynthesis in the algae.

The term ‘gene’ as used herein refers to segment of DNA involved in producing a polypeptide chain. It can include preceding and following the coding region (e.g., 5′ and 3′ untranslated regions, promoters, etc) as well as intervening sequences (introns) between individual coding segments (exons).

The term ‘salicylanilide based drug’ as used herein refers to compounds derived from salicylanilide. The salicylanilide is an amide of salicylic acid and aniline. The derivatives of salicylanilide include halogen functional groups. The derivatives of salicylanilide includes but it is not limited to chlorinated derivative of salicylanilide and brominated derivative of salicylanilide.

The present disclosure relates to novel algal strain or algae. Said algae is mutant algae, resistant to salicylanilide based drug.

In some embodiments of the present disclosure, the mutant algae has improved productivity as compared to wild type of the algae in presence of the salicylanilide based drug.

In some embodiments of the present disclosure, the mutant algae has at least 30% improved productivity as compared to wild type of the algae in presence of the salicylanilide based drug.

In some embodiments of the present disclosure, the mutant algae has about 30% to 100%, including all the values in the range, for instance 30.1%, 30.2%, 30.3%, 30.4% and so on and so forth, improved productivity as compared to wild type of the algae in presence of the salicylanilide based drug.

In some embodiments of the present disclosure, the mutant algae has improved photosynthetic efficiency as compared to wild type of the algae in presence of the salicylanilide based drug.

In some embodiments of the present disclosure, the mutant algae has at least 2 fold improved photosynthetic efficiency as compared to wild type of the algae in presence of the salicylanilide based drug.

In some embodiments of the present disclosure, the mutant algae has about 2 fold to 3 fold improved photosynthetic efficiency as compared to wild type of the algae in presence of the salicylanilide based drug.

In some embodiments of the present disclosure, the mutant algae has at least 2 fold reduced adenosine triphosphate (ATP) depletion as compared to wild type of the algae in presence of the salicylanilide based drug.

In some embodiments of the present disclosure, the mutant algae has about 1.88 fold to 2.1 fold reduced adenosine triphosphate (ATP) depletion as compared to wild type of the algae in presence of the salicylanilide based drug.

In some embodiments of the present disclosure, the mutant algae comprises at least 2 mutations in a gene encoding putative proton symporter protein 3. In embodiment, the putative proton symporter protein 3 having at least 2 mutations is set forth as SEQ ID No. 1. In an embodiment, gene encoding putative proton symporter protein 3 in the wild type algae is set forth as SEQ ID No. 2

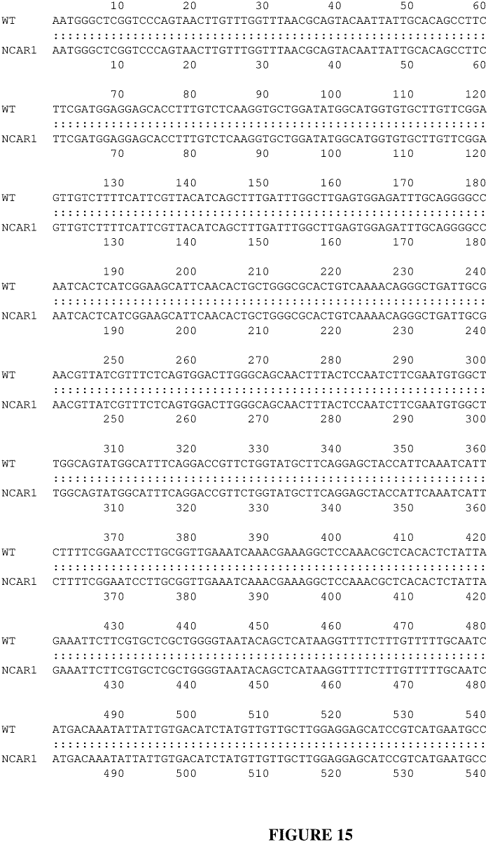

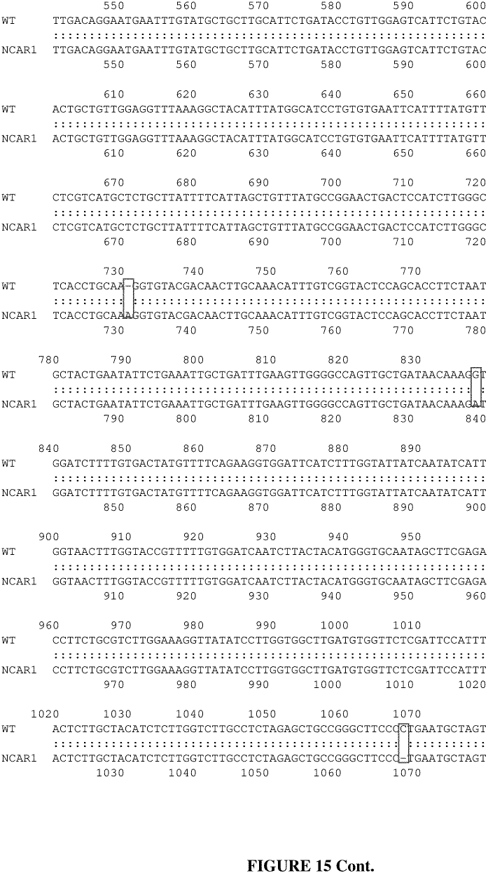

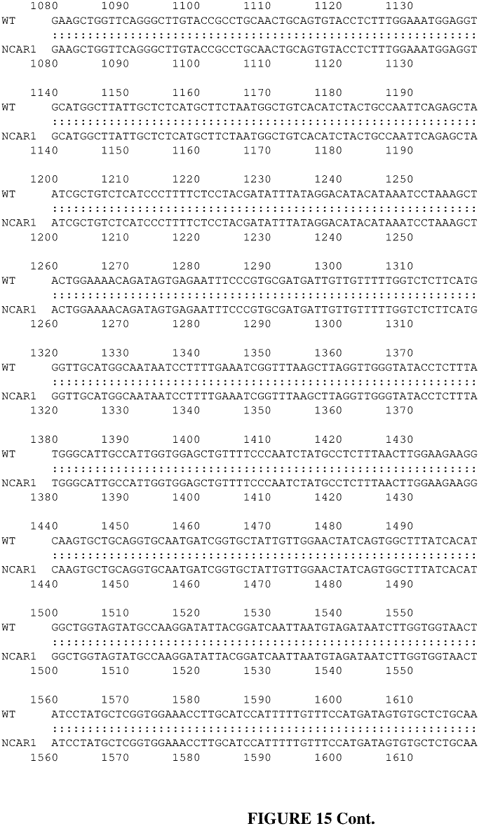

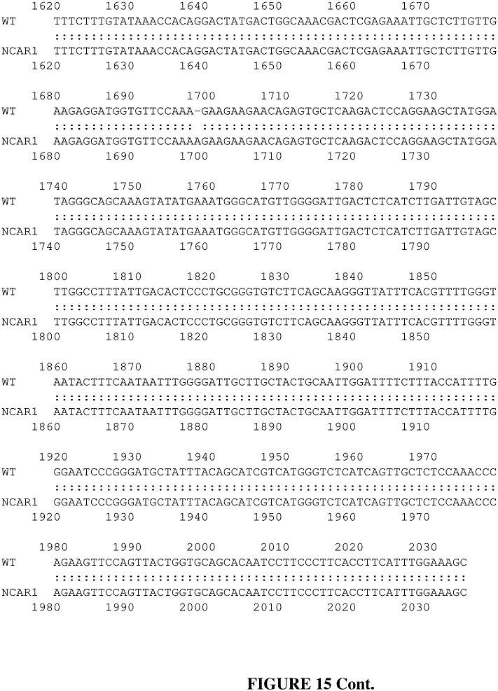

In some embodiments of the present disclosure, the mutant algae comprises 2 mutations in a gene encoding putative proton symporter protein 3. The gene encoding putative proton symporter protein 3 comprises insertion-deletion mutation at about 730 nucleotide position and at about 1070 nucleotide position in the gene. FIG. 15 describes sequence alignment between gene encoding putative proton symporter protein 3 of wild type of the algae and gene encoding putative proton symporter protein 3 of the mutant algae. In the sequence alignment it can be noted that there is insertion of a nucleotide at position 730 and that there is deletion of nucleotide at position 1070 in the mutant algae.

In some embodiments of the present disclosure, the mutant algae comprises at least 1 mutation in a gene encoding putative cytochrome P450 protein 4. In an embodiment, the putative cytochrome P450 protein 4 having at least 1 mutation is set forth as SEQ ID No. 3. In an embodiment, gene encoding putative cytochrome P450 protein 4 in the wild type algae is set forth as SEQ ID No. 4.

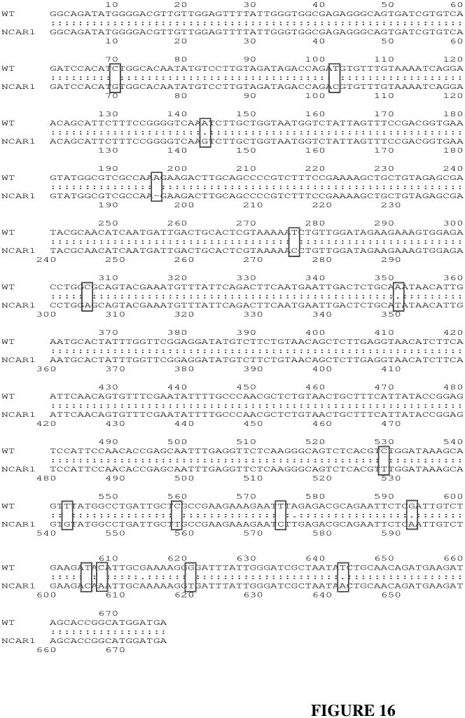

In some embodiments of the present disclosure, the mutant algae comprises 1 mutation in a gene encoding putative cytochrome protein 4. The gene encoding putative cytochrome P450 protein 4 comprises frameshift mutation at about 200 nucleotide position in the gene. FIG. 16 describes sequence alignment between gene encoding putative cytochrome P450 protein 4 of wild type of the algae and gene encoding putative cytochrome P450 protein 4 of mutant algae. In the sequence alignment it can be noted that there is mutation at about 200 nucleotide position in the mutant algae.

In some embodiments of the present disclosure, the mutant algae comprises at least 500 bp insertion in a gene encoding putative aquaporin protein 1 in reading frame. In an embodiment, the gene encoding putative aquaporin protein 1 comprises the nucleotide insertion in at least two positions in the mutant algae. In an embodiment, the putative aquaporin protein 1 of the mutant algae is set forth as SEQ ID No. 5. In an embodiment, gene encoding putative aquaporin protein 1 in wild type algae is set forth as SEQ ID No. 6

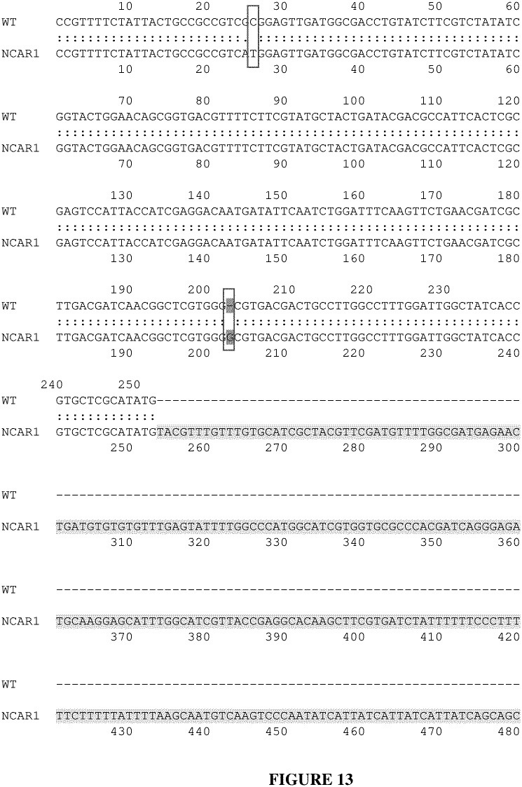

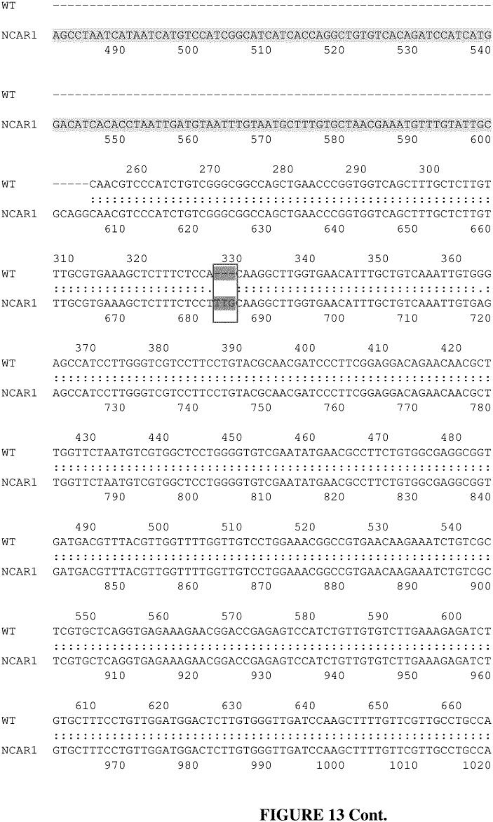

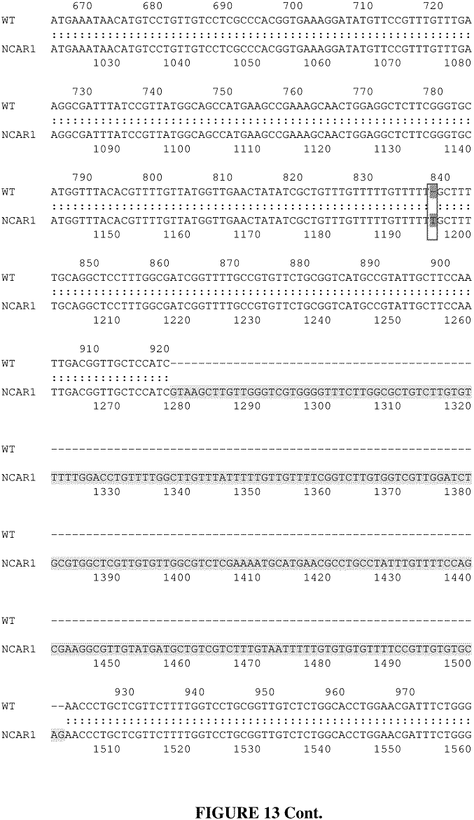

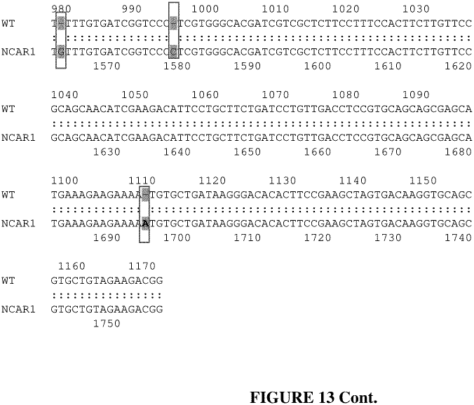

In some embodiments of the present disclosure, the mutant algae comprises about 350 bp insertion at about 255 nucleotide position and about 220 bp insertion at about 1275 nucleotide position, respectively in reading frame of a gene encoding putative aquaporin protein 1 as compared to wild type of the algae. FIG. 13 describes sequence alignment between gene encoding putative aquaporin protein 1 of wild type of the algae and gene encoding putative aquaporin protein 1 of mutant algae. In the sequence alignment it can be noted that there is an insertion at about 255 nucleotide position and another insertion at 1275 nucleotide position in the gene of the mutant algae as compared to wild type of the algae.

In some embodiments of the present disclosure, the mutant algae comprises about 340 bp insertion at about 250 nucleotide position in reading frame of a gene encoding putative aquaporin protein 2. In an embodiment, the gene encoding putative aquaporin protein 2 of the mutant algae is set forth as SEQ ID No. 7. In an embodiment, gene encoding putative aquaporin protein 2 in the wild type algae is set forth as SEQ ID No. 8.

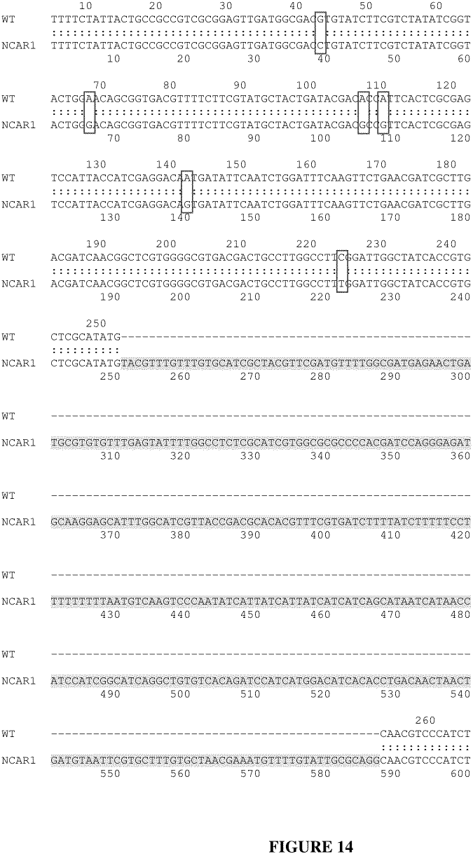

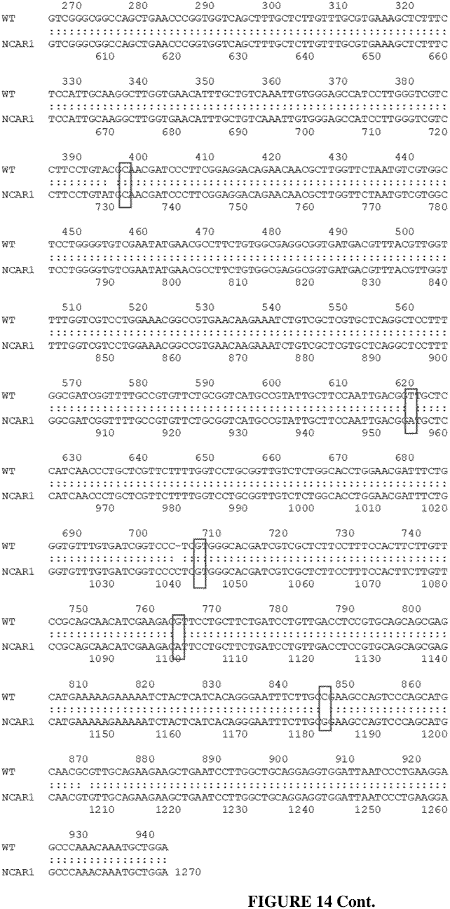

FIG. 14 describes sequence alignment between gene encoding putative aquaporin protein 2 of wild type of the algae and gene encoding putative aquaporin protein 2 of mutant algae. In the sequence alignment it can be noted that there is an insertion at about 250 nucleotide position in the gene of the mutant algae as compared to wild type of the algae.

In some embodiments of the present disclosure, the algae includes but is not limited to Picochlorum sp., Chlorella sp., Nannochloropsis sp., Nannochloris sp. Chlamydomonas sp. Dunaliella sp., Tetraselmis sp., Haematococcus pluvialis, Cyanobacterium sp., Synechocystis sp., and Synechococcus sp.

In some embodiments of the present disclosure, the mutant algae is resistant to salicylanilide based drug by at least 10 times the threshold value as compared to wild type of the algae.

In some embodiments of the present disclosure, the mutant algae is resistant to salicylanilide based drug having concentration ranging from about 0.5 ppm to 10 ppm, including all the values in the range, for instance, 0.6 ppm, 0.7 ppm, 0.8 ppm, 0.9 ppm and so on and so forth, as compared to wild type of the algae.

In some embodiments of the present disclosure, the salicylanilide based drug to which the mutant algae is resistant belong to the Salicylanilide class of drug.

In some embodiments of the present disclosure, the salicylanilide based drug to which the mutant algae is resistant is selected from a group comprising niclosamide, oxyclozanide, rafoxanide, closantel, dibromsalan, metabromsalan, dibromosalicylanilide, tribromosalicylanilide, tetrachlorosalicylanilide, and any combinations thereof.

In some embodiments of the present disclosure, the mutant algae shows improved productivity of at least 30% as compared to wild type of the algae in presence of the salicylanilide based drug.

In some embodiments of the present disclosure, the mutant algae show improved productivity ranging from about 30% to 100%, including all the values in the range, for instance, 31%, 32%, 33%, 34% and so on and so forth, as compared to wild type of the algae in presence of the salicylanilide based drug.

The FIG. 5 describes a plot illustrating improved volumetric productivity (AFDW mg/L/day) of the mutant algae as compared to wild type of the algae at about 1 ppm, about 2 ppm and about 5 ppm of Niclosamide, respectively. The volumetric productivity of the mutant algae in presence of the Niclosamide is equal to or greater than the volumetric productivity of the wild type of the algae in absence of Niclosamide. Thus, clearly establishing that the mutant algae is resistant to salicylanilide based drug, such as Niclosamide as compared to wild type of the algae.

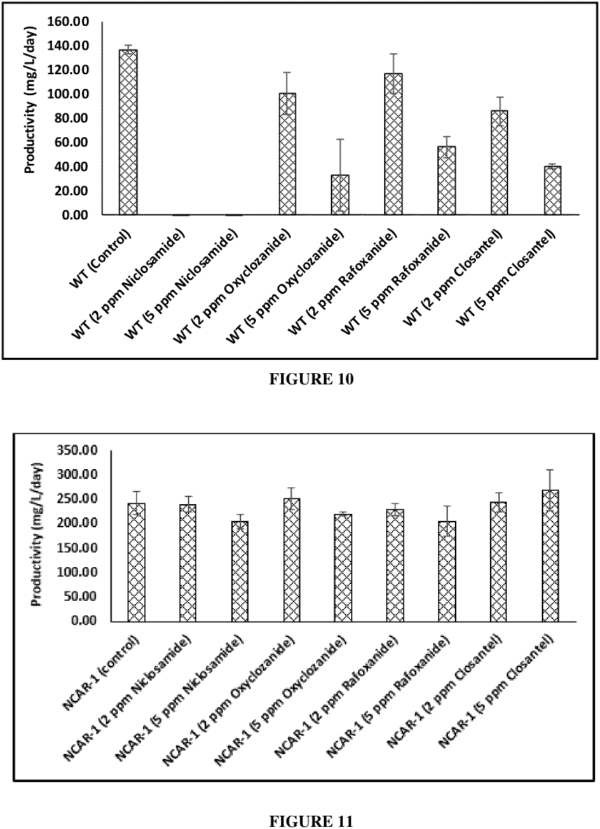

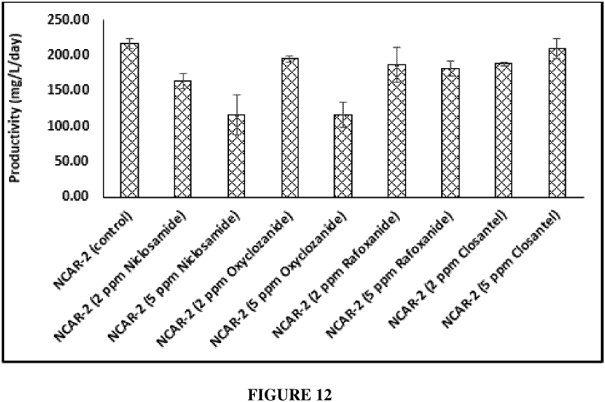

FIGS. 11 and 12 describe plots illustrating improved volumetric productivity (AEDW mg/L/day) of the mutant algae as compared to wild type of the algae (described in FIG. 10) at about 2 ppm and about 5 ppm of Niclosamide, oxyclozanide, Rafoxanide and Closantel, respectively. The plots demonstrate that the mutant algae is resistant to salicylanilide based drug as compared to wild type of the algae.

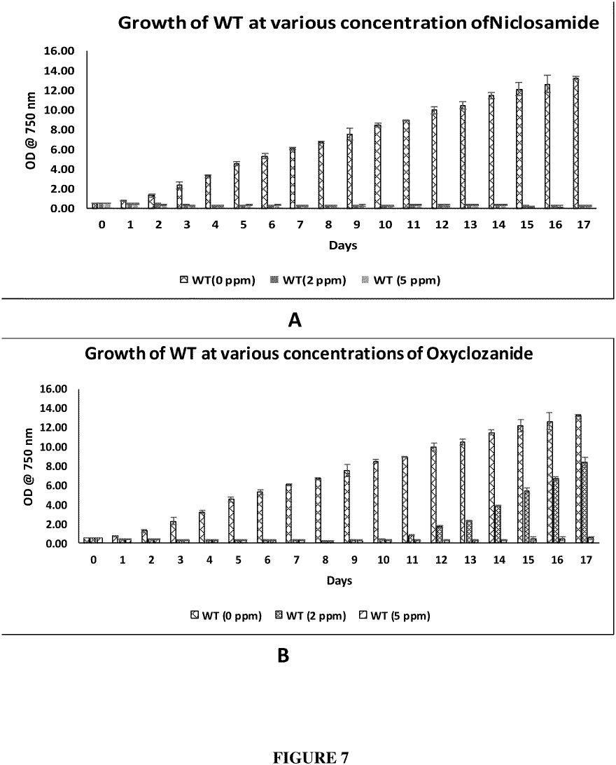

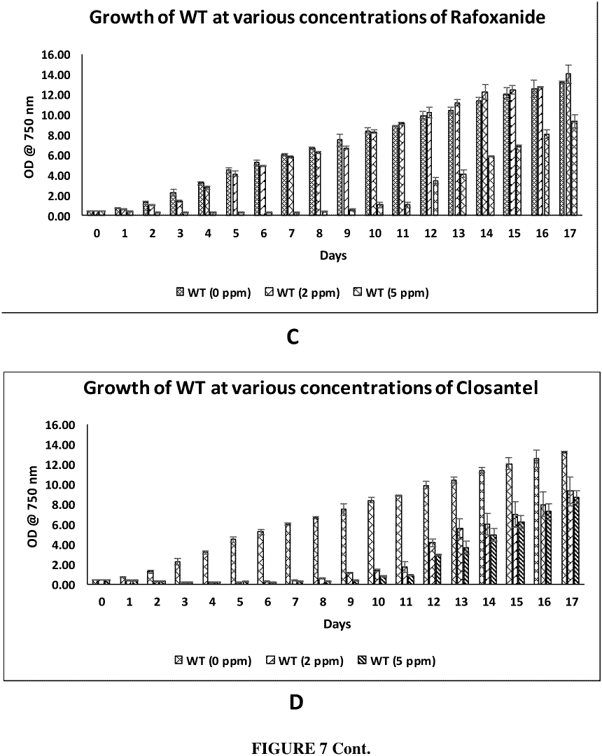

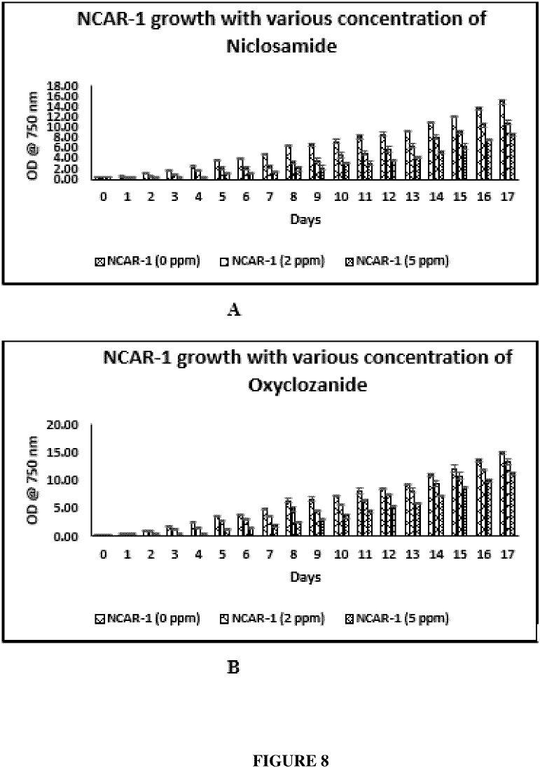

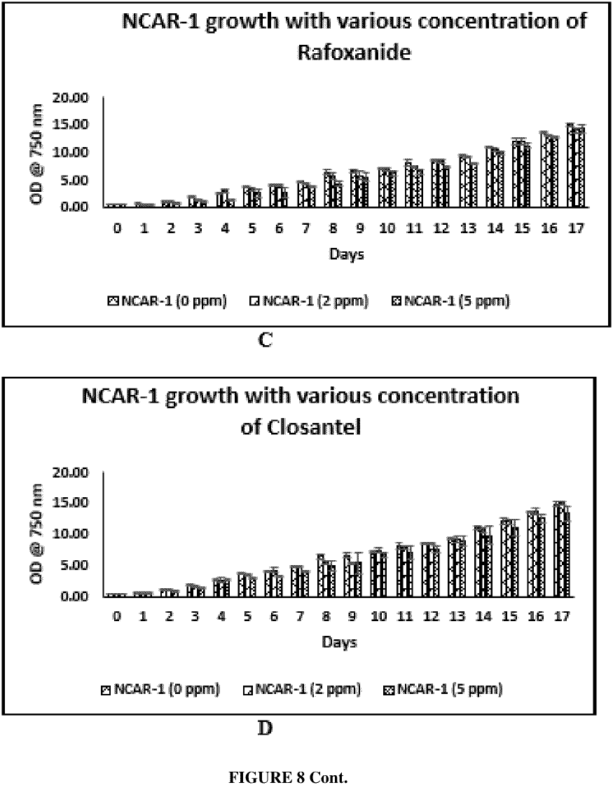

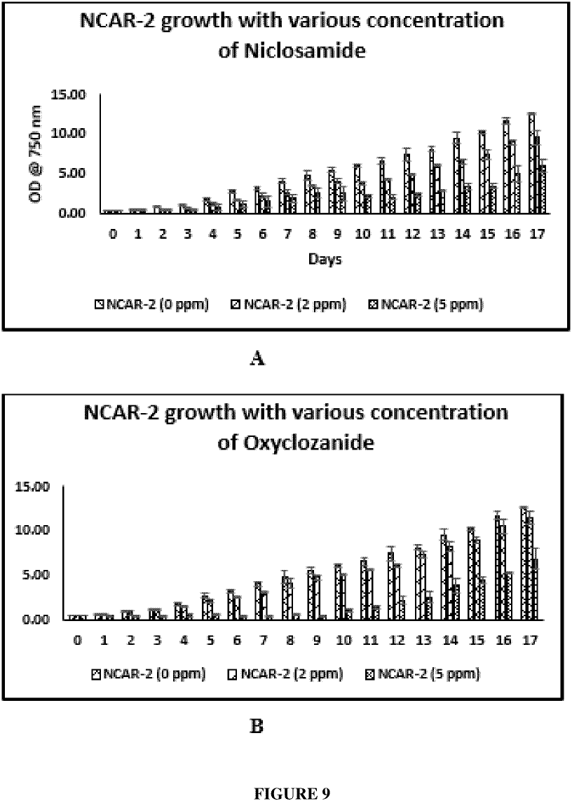

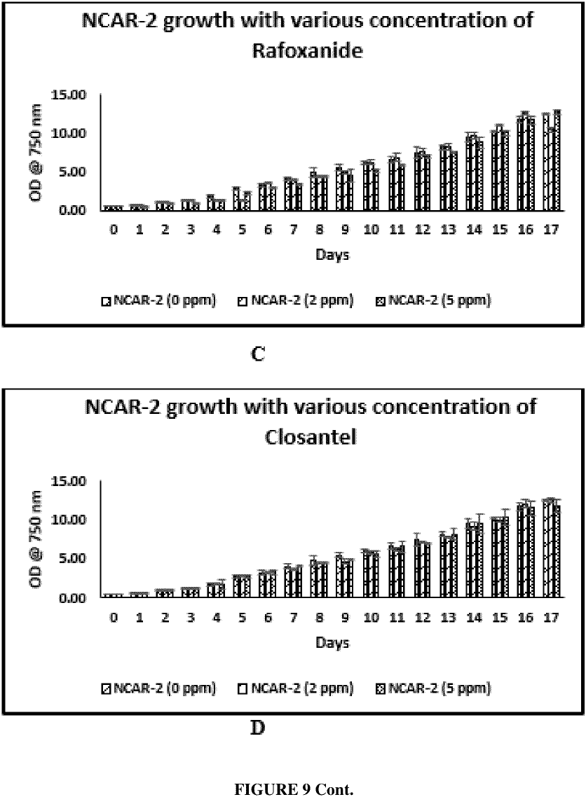

The FIGS. 8 and 9 describe plots illustrating improved growth of the mutant algae as compared to wild type of the algae (described in FIG. 7) at about 2 ppm and about 5 ppm of Niclosamide, Oxyclozanide, Rafoxanide and Closantel, respectively. The plots demonstrate that the mutant algae is resistant to salicylanilide based drug as compared to wild type of the algae.

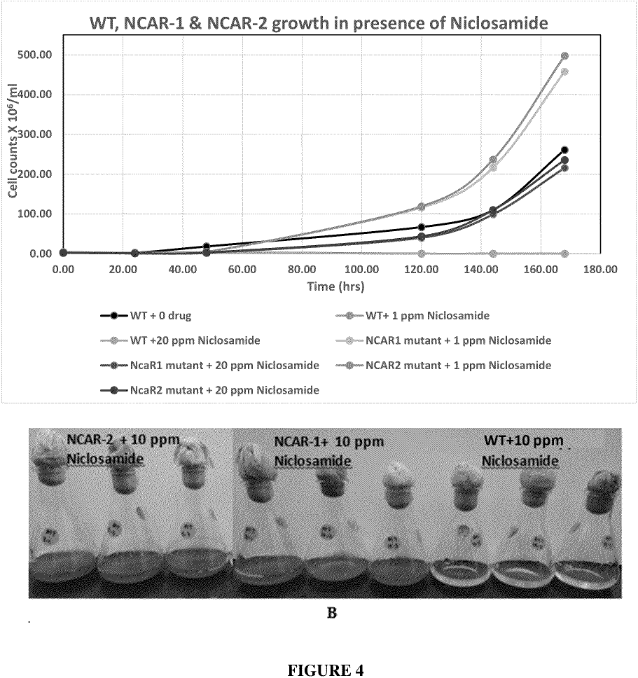

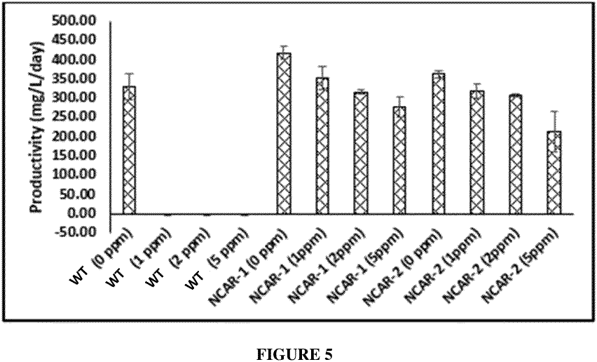

The FIG. 4A describes a plot illustrating improved growth rate of mutant algae in presence of about 10 ppm salicylanilide based drug, such as Niclosamide as compared to wild type of the algae. This is further substantiated by the image under FIG. 4B which shows no growth of wild type of the algae (clear solution) at 10 ppm of Niclosamide. However, the mutant algae show evident growth (green colour solution) at 10 ppm of Niclosamide.

In some embodiments of the present disclosure, the mutant algae show improved photosynthetic efficiency of at least 2 fold as compared to wild type of the algae in presence of the salicylanilide based drug.

In some embodiments of the present disclosure, the mutant algae show improved photosynthetic efficiency ranging from about 2 to 3 as compared to wild type of the algae in presence of the salicylanilide based drug.

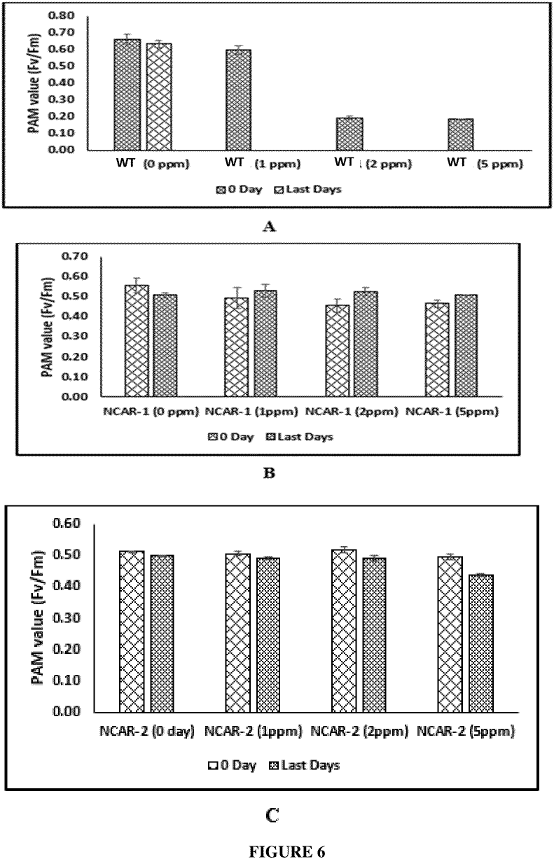

FIG. 6 describes a plot illustrating improved photosynthetic efficiency/health of the mutant algae as compared to wild type of the algae at about 1 ppm, about 2 ppm and about 5 ppm of Niclosamide. The improved photosynthetic efficiency of the mutant algae explicitly implies resistance of the mutant algae to salicylanilide based drug, such as Niclosamide and that the wild type of the algae is not resistant to the salicylanilide based drug.

In some embodiments of the present disclosure, the mutant algae show improved nitrogen content of at least 10% as compared to wild type of the algae in presence of the salicylanilide based drug.

In some embodiments of the present disclosure, the mutant algae shows improved nitrogen content ranging from about 10% to 15%, including all the values in the range, for instance, 10.1%, 10.2%, 10.3%, 10.4% and so on and so forth, as compared to wild type of the algae in presence of the salicylanilide based drug. Carbon, hydrogen, nitrogen and sulfur (CHNS) analysis data of the mutant algae and wild type of the algae is described in Tables 1 and 2.

TABLE 1

| Name of sample | N [%] | C [%] | H [%] | S [%] |
|---|---|---|---|---|
| WT (0 ppm) | 8.42 | 53.09 | 7.74 | 0.96 |
| NCAR- 1 (0 ppm) | 7.21 | 50.57 | 7.66 | 0.77 |
| NCAR-1 (1 ppm) | 8.08 | 51.1 | 7.68 | 0.82 |
| NCAR-1 (2 ppm) | 8.82 | 51.58 | 7.82 | 0.88 |
| NCAR-1 (5 ppm) | 9.25 | 51.32 | 7.68 | 0.86 |
| NCAR-2 (0 ppm) | 7.98 | 51.47 | 7.73 | 0.76 |
| NCAR-2 (1 ppm) | 8.05 | 51.66 | 7.75 | 0.76 |
| NCAR-2 (2 ppm) | 8.46 | 51.81 | 7.63 | 0.79 |
| NCAR-2 (5 ppm) | 8.85 | 50.89 | 7.67 | 0.79 |

The data in Table 1 shows that the mutant algae has improved nitrogen content in presence of salicylanilide based drug, such as Niclosamide. The nitrogen content is found to be about 10% to 14% higher in the mutant algae as compared to the nitrogen content observed in wild type of the algae grown in absence of salicylanilide based drug. This data explicitly demonstrates that the mutant algae has improved growth rate in presence of salicylanilide based drug as compared to wild type of the algae.

Further, the data in Table 2 shows that the mutant algae has improved nitrogen content in presence of salicylanilide based drug, such as Niclosamide, Oxyclozonide, Rafoxanide and Closantel, respectively. The data shows that the nitrogen content was about 10% to 15% higher in the mutant algae in presence of the salicylanilide based drug as compared to wild type of the algae.

TABLE 2

| | Name of sample | N [%] | C [%] | H [%] | S [%] |
|---|---|---|---|---|---|
| 1 | Wild type (WT) (Control) | 8.33 | 49.64 | 7.28 | 1.09 |
| 2 | WT (2 ppm Oxyclozanide) | 9.63 | 48.23 | 7 | 1.01 |
| 3 | WT (5 ppm Oxyclozanide) | 10.22 | 46.23 | 6.7 | 0.9 |
| 4 | WT (2 ppm Rafoxanide) | 8.87 | 47.27 | 7.07 | 1.04 |
| 5 | WT (5 ppm Rafoxanide) | 9.22 | 48.31 | 7.17 | 1.01 |
| 6 | WT (2 ppm Closantel) | 7.82 | 48.29 | 7.2 | 1 |
| 7 | WT (5 ppm Closantel) | 9.07 | 47.94 | 7.15 | 0.98 |
| 8 | NCAR-1 (control) | 6.09 | 46.12 | 7.16 | 0.72 |
| 9 | NCAR-1 (2 ppm Niclosamide) | 7.88 | 46.86 | 7.16 | 0.8 |
| 10 | NCAR-1 (5 ppm Niclosamide) | 8.28 | 46.88 | 7.18 | 0.83 |
| 11 | NCAR-1 (2 ppm Oxyclozanide) | 6.41 | 46.7 | 7.13 | 0.76 |
| 12 | NCAR-1 (5 ppm Oxyclozanide) | 7.46 | 47.39 | 7.13 | 0.83 |
| 13 | NCAR-1 (2 ppm Rafoxanide) | 6.62 | 46.72 | 7 | 0.76 |
| 14 | NCAR-1 (5 ppm Rafoxanide) | 6.91 | 47.61 | 7.02 | 0.75 |
| 15 | NCAR-1 (2 ppm Closantel) | 6.49 | 47.44 | 7.05 | 0.74 |
| 16 | NCAR-1 (5 ppm Closantel) | 6.8 | 47.23 | 7.06 | 0.76 |
| 17 | NCAR-2 (control) | 7.19 | 47.88 | 7.19 | 0.74 |
| 18 | NCAR-2 (2 ppm Niclosamide) | 8.12 | 45.53 | 7.02 | 0.81 |
| 19 | NCAR-2 (5 ppm Niclosamide) | 9.37 | 46.84 | 6.99 | 0.8 |
| 20 | NCAR-2 (2 ppm Oxyclozanide) | 7.47 | 47.21 | 7.15 | 0.75 |
| 21 | NCAR-2 (5 ppm Oxyclozanide) | 8.87 | 46.87 | 7.14 | 0.78 |
| 22 | NCAR-2 (2 ppm Rafoxanide) | 6.57 | 46.94 | 7.2 | 0.72 |
| 23 | NCAR-2 (5 ppm Rafoxanide) | 7.61 | 47.12 | 7.23 | 0.77 |
| 24 | NCAR-2 (2 ppm Closantel) | 6.94 | 46.73 | 7.18 | 0.74 |
| 25 | NCAR-2 (5 ppm Closantel) | 7.17 | 47.42 | 7.24 | 0.76 |

The disclosure further describes a method of obtaining the mutant algae described above.

In some embodiments of the present disclosure, the method of obtaining the mutant algae comprises:
subjecting wild type of the algae to mutagenesis;
culturing mutagen exposed live algae under dark and subsequently in low light conditions to obtain modified algae; and
enriching the modified algae to obtain the algae resistant to salicylanilide based drug.

In some embodiments of the present disclosure, the mutagenesis is carried out by exposing the wild type of the algae to Alkylating agent, such as ethyl methanesulfonate (EMS). In an embodiment, the mutant algae can be obtained by exposing the wild type algae to mutagenic chemicals such as methyl methanesulfonate (MMS), N-methyl-N-nitrosourea, vinyl chloride, Methylhydrazine, Busulfan, Carmustine, lomustine, Dimethyl sulfate, Temorzolomide, Dacarbazine, 5-bromouracil, 2-aminopurine, Ethidium bromide, proflavine, acridine orange, Nickel, Chromium, Cobalt, Cadmium, Arsenic, All these mutagenic chemicals can induce point mutations in DNA and hence generate the mutant algae described above. In another embodiment, Physical mutagens, such as, UV radiation, X rays, gamma rays and particle radiation, such as fast and thermal neutrons, beta and alpha particles can be employed for obtaining the mutant algae.

In some embodiments of the present disclosure, the alkylating agent, such as ethyl methanesulfonate is at a concentration ranging from about 0.4 M to 1 M. In an embodiment, the mutagen, such as chemical mutagen is at a concentration of about 0.4 M, about 0.5 M, about 0.6 M, about 0.7 M, about 0.8 M, about 0.9 M or about 1 M.

In some embodiments of the present disclosure, the mutagenic chemicals are at a concentration ranging from about 0.4 M to 1 M. In an embodiment, the mutagen, such as chemical mutagen is at a concentration of about 0.4 M, about 0.5 M, about 0.6 M, about 0.7 M, about 0.8 M, about 0.9 M or about 1 M.

In some embodiments of the present disclosure, mutagenesis is carried out for a duration ranging from about 1 hours to 2 hours. In an embodiment, the mutagenesis is carried out for a duration of about 1 hour, about 1.1 hours, about 1.2 hours, about 1.3 hours, about 1.4 hours, about 1.5 hours, about 1.6 hours, about 1.7 hours, about 1.8 hours, 1.9 hours or about 2 hours.

In some embodiments of the present disclosure, the culturing described in the method comprises:
holding mutagen exposed live algae cells under dark for a duration ranging from about 12 hours to 18 hours; and
growing the algae in low light having intensity ranging from about 100 µmoles/s/m² to 400 µmoles/s/m² for a duration ranging from about 7 days to 14 days to obtain modified algae.

In an embodiment, the mutagen exposed live algae cells are held under dark for a duration ranging from about 12 hours to 18 hours, including all the values in the range, for instance, 12.1 hours, 12.2 hours, 12.3 hours, 12.4 hours and so on and so forth.

In an embodiment, after holding the live algae cells in dark, the algae were allowed to grow in low light having intensity ranging from about 100 µmoles/s/m² to 400 µmoles/s/m², including all the values in the range, for instance, 101 µmoles/s/m², 102 µmoles/s/m², 103 µmoles/s/m², 104 µmoles/s/m² and so on and so forth. In an embodiment, the live algae cells are exposed to said low light for a duration of about 7 days, about 8 days, about 9 days, about 10 days, about 11 days, about 12 days, about 13 days or about 14 days.

In some embodiments of the present disclosure, the culturing is carried out at a temperature ranging from about 28° C. to 35° C., including all the values in the range, for instance, 28.5° C., 29° C., 29.5° C., 30° C. and so on and so forth. In an embodiment, the culturing is carried out in a culture medium including but not limited to urea and phosphoric acid. In an embodiment, the culturing is carried out in a culture medium having pH ranging from about 6.8 to 7.5, including all the values in the range, for instance, 6.85, 6.90, 6.95, 7.00 and so on and so forth.

In some embodiments of the present disclosure, enriching the modified algae comprises-exposing the modified algae to salicylanilide based drug including but not limited to niclosamide, oxyclozanide, rafoxanide, closantel, dibromsalan, metabromsalan, dibromosalicylanilide, tribromosalicylanilide, tetrachlorosalicylanilide, and any combinations thereof, followed by propagating the algae for at least 300 generations, preferably for about 300 to 350 generations, for a duration ranging from about 300 days to 370 days, including all the values in the range, for instance, 301 days, 302 days, 303 days, 304 days and so on and so forth, to obtain mutant algae resistant to salicylanilide based drug. The propagation of the algae is carried out until constant growth of the algae is achieved as compared to wild type of the algae.

In some embodiments of the present disclosure, the enriching is carried out by exposing the modified algae to increasing dosage of the salicylanilide based drug (e.g., about 0.5 ppm to about 1 ppm to about 2 ppm) after certain interval of growth at a fixed concentration. After selection at each concentration, growth rate of the algae is assessed and subsequently the concentration of the salicylanilide based drug is increased by at least 0.5 ppm. For instance, if the enriching of the algae is initiated at 0.5 ppm, upon checking the growth rate of the algae at a duration of about 100 days, salicylanilide based drug is increased by a concentration of about 0.5 ppm and so on and so forth.

In some embodiments of the present disclosure, the method comprises subjecting the propagated algae to Fluorescence-activated cell sorting (FACS) to separate mutant algae resistant to salicylanilide based drug from algae not resistant to salicylanilide based drug. The parameter for FACS sorting is forward-angle light scatter (FSC) and side-angle scatter (SSC) measurements concurrently to sort out cells with a size range of about 2 µ to about 3 µ.

In some embodiments of the present disclosure, the method of obtaining the mutant algae comprises:
subjecting the wild type algae to mutagenesis by exposing the algae to mutagen including but not limited to ethyl methanesulfonate (EMS) for a duration ranging from about 1 hour to 2 hours;
holding the mutagen exposed live algae under dark conditions for a duration ranging from about 12 hours to 18 hours, followed by culturing under low light condition having intensity ranging from about 100 µmoles/s/m²to 400 µmoles/s/m², for at least a week to obtain modified algae;
enriching the modified algae by exposing the algae to at an increased concentration of salicylanilide based drug, wherein concentration of salicylanilide based drug is increased upon assessing growth rate of the algae, followed by propagating the algae for at least 300 generation to obtain mutant algae resistant to salicylanilide based drug; and
sorting the mutant algae resistant to salicylanilide based drug from the algae not resistant to salicylanilide based drug.

In some embodiments of the present disclosure, the method further comprises growth screening of the obtained mutant algae to obtain stable mutant algae. For instance, the obtained mutant algae is subjected to one or more rounds of growth screening, wherein the mutant algae is checked for growth in presence of salicylanilide based drug. In some embodiments, the obtained mutant algae are subjected to at least 10 rounds of growth screening in presence of salicylanilide based drug to obtain stable mutant algae.

In some embodiments, the mutant algae described in the present disclosure is stable mutant algae resistant to salicylanilide based drug as compared to wild type of the algae.

The mutant algae and the method of obtaining the mutant algae described in the present disclosure provides for following advantages-
The mutant algae enable use of the salicylanilide based drug as a crop control agent during outdoor pond cultivation and PBR cultivation of the algae on large scale without affecting biomass productivity of the algae.
The mutant algae and use of the salicylanilide based drug during outdoor pond cultivation or PBR cultivation of algae helps in generation of high nitrogen (high protein) containing biomass thus maximizing Nitrogen use efficiency.
The mutant algae enable stable round the year outdoor pond and indoor PBR cultivation with consistent biomass production achieved due to effective crop protection against predators or grazers.
The mutant algae enable production of high quality biomass with high nitrogen content which increases biomass values and maximizes nitrogen use from the media and environment.

The mutant algae of the present disclosure having all the characteristic features that are described above is deposited at National Agriculturally Important Microbial Culture Collection (NAIMCC). Date of submission-15^{th} December 2021. Following are the accession numbers-NAIMCC-IDA-1 and NAIMCC-IDA-2.

It is to be understood that the foregoing description is illustrative not a limitation. While considerable emphasis has been placed herein on particular features of this disclosure, it will be appreciated that various modifications can be made, and that many changes can be made in the preferred embodiments without departing from the principles of the disclosure. Those skilled in the art will recognize that the embodiments herein can be practiced with modification within the spirit and scope of the embodiments as described herein. Similarly, additional embodiments and features of the present disclosure will be apparent to one of ordinary skill in art based upon description provided herein.

Descriptions of well-known/conventional methods/steps and techniques are omitted so as to not unnecessarily obscure the embodiments herein. Further, the disclosure herein provides for examples illustrating the above-described embodiments, and in order to illustrate the embodiments of the present disclosure, certain aspects have been employed. The examples used herein for such illustration are intended merely to facilitate an understanding of ways in which the embodiments may be practiced and to further enable those of skill in the art to practice the embodiments. Accordingly, following examples should not be construed as limiting the scope of the embodiments herein.

### EXAMPLES

### Example 1: Preparation of Mutant Algae

About 1 × 10⁸ /ml WT cells were treated with 0.6 M EMS for about 1 hours. EMS exposure resulted in about 50% killing of the WT cells. The alive cells were allowed to recover in dark overnight and then in low light for about a week. Upon recovering the mutant algae population was exposed to about 0.5 ppm of Niclosamide and enriched in presence of Niclosamide for several generations. As the growth rate in the presence of Niclosamide increased, the selection pressure was increased to about 1 ppm and then to about 2 ppm of Niclosamide, respectively. The mutant pool was grown in presence of about 2 ppm Niclosamide (for about 300 generations, about 1 year) till the growth rates became constant. At this stage, FACS sorting of the Niclosamide resistant mutant pool was performed to get individual Niclosamide resistant mutant algae. About 100 individual mutants were isolated and were further checked for growth in the presence of about 2 ppm Niclosamide. The list was narrowed down to about 10 stable mutants after about 2 rounds of growth screening. Finally, after about 10 rounds of repeated growth screening in the presence of about 2 ppm Niclosamide, stable mutant algae (NCAR-1 & NCAR-2) were selected, based on the stable drug resistant phenotype and consistent growth & productivity.

### Example 2: Measuring ATP Levels in Mutant Algae and Wild Type of the Algae

Total intracellular ATP levels in wild type algae and the mutant algae were measured using molecular probes bioluminescence assay kit. The assay is based on luciferase’s requirement for ATP in producing light (emission maximum of about 560 nm at pH 7.8) from the reaction

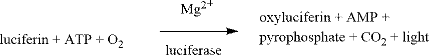

Niclosamide was added to mid log cells (OD₇₅₀ at about 0.3) of both wild type algae and mutant algae and samples (OD = 10⁸ cells) were collected after about 12 hours for ATP measurement. About 1 ppm of Oligomycin A (inhibitor of ATP synthase) was used as positive control to inhibit ATP synthesis. intracellular ATP concentration in mutant algae and wild type of the algae in presence of Niclosamide was described in Table 3.

TABLE 3

| | | | | | |
|---|---|---|---|---|---|
| Name of the sample | 1 | 2 | Average | SD | ATP conc.(nM) |
| Wild type (WT) | 1833 | 1613 | 1723 | 155.56 | ~ 284.37 |
| WT + 1 ppm Oligomycin | 1181 | 982 | 1081.5 | 140.71 | 178.49 |
| WT + 1 ppm Niclosamide | 204 | 145 | 174.5 | 41.72 | ~ 28.80 |
| NCRA-1 | 1633 | 1569 | 1601 | 45.25 | 264.24 |
| NCAR-1 + 1 ppm Niclosamide | 835 | 688 | 761.5 | 103.94 | 125.68 |
| NCRA 2 | 1613 | 1549 | 1581 | 34.65 | 261 |
| NCRA-2 + 1 ppm Niclosamide | 890 | 789 | 839.5 | 101 | 138.55 |

Observations:
Oligomycin A (about 1 ppm) addition leads to about 40% lower ATP levels in about 12 hours in wild type (WT) of the algae.
Niclosamide (about 1 ppm) addition leads to about 90% depletion of ATP in about 12 hours in wild type (WT) of the algae.
In mutant algae (NCAR-1 and NCAR-2), the ATP depletion was about 50% after about 12 hours of salicylanilide based drug addition. This demonstrates increased resistant of the mutant algae to salicylanilide based drug as compared to wild type of the algae.

The foregoing description of the specific embodiments reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments in this disclosure have been described in terms of preferred embodiments, those skilled in the art will recognize that the embodiments herein can be practiced with modification within the spirit and scope of the embodiments as described herein.

Throughout this specification, the term ‘combinations thereof’ or ‘any combination thereof’ or ‘any combinations thereof’ a are used interchangeably and are intended to have the same meaning, as regularly known in the field of patents disclosures.

As regards the embodiments characterized in this specification, it is intended that each embodiment be read independently as well as in combination with another embodiment. For example, in case of an embodiment 1 reciting 3 alternatives A, B and C, an embodiment 2 reciting 3 alternatives D, E and F and an embodiment 3 reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Any discussion of documents, acts, materials, devices, articles and the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

We claim:

1. Mutant algae resistant to salicylanilide based drug having improved productivity and improved photosynthetic efficiency as compared to wild type of the algae in presence of salicylanilide based drug.

2. The algae as claimed in claim 1, wherein the algae comprises at least 2 mutations in a gene encoding putative proton symporter protein 3.

3. The algae as claimed in claim 1, wherein the algae comprises at least 1 mutation in a gene encoding putative cytochrome p450 protein 4.

4. The algae as claimed in claim 1, wherein the algae comprises at least 500 bp insertion in a gene encoding putative aquaporin protein 1.

5. The algae as claimed in claim 1, wherein the algae comprises at least 300 bp insertion in a gene encoding putative aquaporin protein 2.

6. The algae as claimed in claim 2, wherein the gene encoding putative proton symporter protein 3 comprises insertion-deletion mutation at about 730 nucleotide position and at about 1070 nucleotide position in the gene.

7. The algae as claimed in claim 3, wherein the gene encoding putative cytochrome p450 protein 4 comprises frameshift mutation at about 200 nucleotide position.

8. The algae as claimed in claim 1, wherein the algae is a microalgae selected from a group comprising picochlorum sp., chlorella sp., nannochloropsis sp., nannochloris sp. Chlamydomonas sp. Dunaliella sp., tetraselmis sp., haematococcus pluvialis, cyanobacterium sp., synechocystis sp., and synechococcus sp.

9. The algae as claimed in claim 1, wherein the algae is resistant to the salicylanilide based drug by at least 10 times threshold value as compared to wild type of the algae.

10. The algae as claimed in claim 1, wherein the algae is resistant to the salicylanilide based drug having concentration ranging from about 0.5 ppm to 10 ppm.

11. The algae as claimed in claim 1, wherein the productivity is improved by at least 30% as compared to wild type of the algae; or wherein the photosynthetic efficiency is improved by at least 2 fold as compared to wild type of the algae.

12. (canceled)

13. The algae as claimed in claim 1, wherein nitrogen content in the algae is improved by at least 10 % as compared to wild type of the algae.

14. The algae as claimed in claim 1, wherein the algae has at least 2 fold reduced adenosine triphosphate (atp) depletion as compared to wild type of the algae.

15. The algae as claimed in claim 1, wherein the salicylanilide based drug to which the algae is resistant is selected from a group comprising niclosamide, oxyclozanide, rafoxanide closantel, dibromsalan, metabromsalan, dibromosalicylanilide, tribromosalicylanilide, tetrachlorosalicylanilide, and combinations thereof.

16. A method of obtaining the mutant algae as claimed in claim 1, said method comprises:
subjecting wild type algae to mutagenesis;
culturing mutagen exposed live algae under dark and subsequently in low light conditions to obtain modified algae; and
enriching the modified algae to obtain the mutant algae resistant to salicylanilide based drug.

17. The method as claimed in claim 16, wherein the mutagenesis is carried out by exposing the wild type of the algae to mutagen selected from a group ethyl methanesulfonate (ems) methyl methanesulfonate (mms), n-methyl-n-nitrosourea, vinyl chloride, methylhydrazine, busulfan, carmustine, lomustine, dimethyl sulfate, temozolomide, dacarbazine, 5-bromouracil, 2-aminopurine, ethidium bromide, proflavine, acridine orange, nickel, chromium, cobalt, cadmium, arsenic, uv radiation, x rays, gamma rays and particle radiation, such as fast and thermal neutrons, beta and alpha particles and wherein the mutagen is at a concentration ranging from about 0.4 m to 1 m.

18. The method as claimed in claim 16, where the culturing involves holding the mutagen exposed live algae under dark for a duration ranging from about 12 hours to 18 hours and subsequently culturing the algae in low light having intensity raging from about 100 µ moles/s/m2 to 400 µ moles/s/m2for a duration ranging from about 7 days to 14 days.

19. The method as claimed in claim 16, wherein the enriching the modified algae comprises exposing the modified algae to salicylanilide based drug, followed by propagating the algae for about 300 generations to 350 generations for a duration ranging from about 300 days to 370 days.

20. The method as claimed in claim 19, wherein the propagation of the algae is carried out till constant growth rate of the algae is achieved as compared to wild type of the algae.

21. The method as claimed in claim 19, wherein the method further comprises subjecting the propagated algae to fluorescence-activated cell sorting (facs) to obtain the algae resistant to salicylanilide based drug.
